(19) United States
(12) Patent Application Publication
(10) Pub. No.: US 2007/0215158 A1
(43) Pub. Date: Sep. 20, 2007

(54) Mask restraining system

(75) Inventors: Kevin D. Kroupa, Ballwin, MO (US); William Kullmann, Oakville, MO (US); Stephen Palmer, Kirkwood, MO (US)

Correspondence Address: GREENSFELDER HEMKER & GALE PC, SUITE 2000, 10 SOUTH BROADWAY, ST LOUIS, MO, 63102, US

(73) Assignee: Allied Healthcare Products, Inc., St. Louis, MO (US)

(21) Appl. No.: 11/377,092

(22) Filed: Mar. 16, 2006

(51) Int. Cl. A61M 16/00; A62B 18/08

(52) U.S. Cl. 128/205.13; 128/207.11

(57) ABSTRACT

A mask restraining system used with a bag mask resuscitator for securely seating a mask on a patient's face is disclosed. The mask restraining system may include a retainer defining upper and lower slots adapted to engage first and second cords, respectively. The bag mask resuscitator may include hollow, flexible tubing having a distal end in communication with a resuscitation bag for forcing air through the flexible tubing and a proximal end in communication with a valve assembly that is rotatably engaged with the mask. The mask includes a port adapted to engage an aperture defined by the retainer in order to engage the mask to the retainer. The first and second cords may be engaged to the upper slots and lower slots, respectively, in order to securely seat the mask on the patient' face when the first and second cords are tightened relative to the retainer.

### FIELD

The present document relates to a mask restraining system used with a bag mask resuscitator.

### SUMMARY

In an embodiment, the mask restraining system comprises a retainer, the retainer having a retainer body defining an aperture, the retainer body further defining an upper portion and a lower portion, the upper portion defining a pair of upper slots, while the lower portion defines a pair of lower slots; and a first cord and a second cord each being adapted to engage the retainer body, wherein the upper slots define a progressively narrowing pinch wedge adapted to engage either the first cord or the second cord in a pinching action and the lower slots define a progressively narrowing pinch wedge adapted to engage another of the first cord or the second cord in a pinching action.

In another embodiment, a resuscitator apparatus comprises a resuscitation bag in fluid flow communication with a mask seated on the face of a patient, wherein the mask defines a port, and a mask restraining system adapted to engage the mask in a tightening action, the mask restraining system comprising a retainer, the retainer including a retainer body defining an aperture, the aperture being adapted to engage the port of the mask, the retainer body further defining an upper portion and a lower portion, the upper portion defining a pair of upper slots, while the lower portion defines a pair of lower slots, and a first cord and a second cord, wherein the upper slots are adapted to engage the first cord in a pinching action while the lower slots are adapted to engage the second cord in a pinching action such that a tightening action is applied to the mask by the retainer body.

In one embodiment, a method of using a mask restraining system comprises:

a) providing a mask restraining system comprising a retainer, the retainer comprising a retainer body defining an aperture, the retainer body further defining an upper portion and a lower portion, the upper portion defining a pair of upper slots, while the lower portion defines a pair of lower slots, the upper slots each defining a progressively narrowing pinch wedge and the lower slots each defining an entry portion in communication with a progressively narrowing pinch wedge, the upper slots adapted to engage a first cord having a middle portion defined between opposite end portions and the lower slots adapted to engage a second cord having a middle portion defined between opposite end portions;

b) engaging the port of the mask with the aperture of the retainer body;

c) passing the first cord and the second cord behind the neck of a patient;

d) engaging the end portions of the first cord to the upper slots and engaging the end portions of the second cord to the lower slots; and

e) pulling the end portions of the first cord and the second cord away from the retainer body such that the first cord and the second cord are engaged to the upper slots and the lower slots, respectively, in a pinching action.

In yet another embodiment, a mask restraining system comprises a retainer, the retainer comprising a retainer body defining an aperture, the retainer body defining an upper portion and a lower portion, the upper portion defining a pair of upper slots, while the lower portion defines a pair of lower slots, each of the upper slots defining a progressively narrowing pinch wedge while each of the lower slots define an entry portion in communication with a progressively narrowing pinch wedge, the upper slots being adapted to frictionally engage a first cord and the lower slots being adapted to frictionally engage a second cord.

In a further embodiment, a mask restraining system comprises a retainer, the retainer comprising a retainer body defining an aperture, the retainer body further defining an upper portion and a lower portion, the upper portion defines a pair of upper slots, while the lower portion defines a pair of lower slots; and a first cord and a second cord, the first cord and the second cord being adapted engage the upper slots and the lower slots, respectively, in a pinching action.

Implementation of the above embodiments may include one or more or more of the following features:

The upper slots define a pair of opposite single angled slots with each of the opposite single angled slots being in communication with an entrance.

The lower slots define a pair of opposite double angled slots with each of the pair of opposing double angled slots being in communication with an entrance.

Each of the entrances define a protrusion.

The first cord and second cord are a bungee cord.

The first cord and the second cord include opposite end portions wherein each of the opposite end portions is adapted to engage the upper slots and the lower slots in a manner that permits the middle portions of the first cord and the second cord to be tightened towards the retainer body.

The opposite single angled slots define the progressively narrowing pinch wedge.

The opposite double angle slots include an entry portion in communication with the progressively narrowing pinch wedge.

The entry portion is at a different angle relative to the progressively narrowing wedge.

The first cord and a second cord include opposite end portions, wherein each of the opposite end portions is adapted to engage the upper slots and the lower slots in a manner that permits the middle portions of the first cord and the second cord to be tightened towards the retainer body.

The opposite single angled slots define a progressively narrowing pinch wedge.

The opposite double angle slots include an entry portion in communication with a progressively narrowing pinch wedge.

The entry portion is at a different angle relative to the progressively narrowing wedge.

The step of positioning the first cord and the second cord behind the bridge of the neck of the patient includes positioning the middle portion of each of the first cord and the second cord behind the bridge of the neck of the patient

Additional features will be set forth in the description which follows or will become apparent to those skilled in the art upon examination of the drawings and detailed description which follows.

### BRIEF DESCRIPTION OF THE DRAWINGS

FIG. 1 is side view of a bag mask resuscitator;

FIG. 2 is a perspective view of a mask restraining system attached to the bag mask resuscitator;

FIG. 3 is perspective view of the mask restraining system attached to a mask of the bag mask resuscitator;

FIG. 4 is a side view of the mask restraining system attached to the mask of the bag mask resuscitator;

FIG. 5 is a top plan view of a retainer of the mask restraining system;

FIG. 6 is a perspective view of the mask of the bag mask resuscitator;

FIG. 7 is a side view of the first and second cords of the mask restraining system;

FIG. 7A is an enlarged view of the first and second cords shown in FIG. 7;

FIG. 7B is another enlarged view of the first and second cords shown in FIG. 7; and

FIGS. 8A-8D illustrate the sequence of attaching the mask restraining system to the mask of the bag mask resuscitator.

Corresponding reference characters indicate corresponding elements among the view of the drawings.

### DETAILED DESCRIPTION

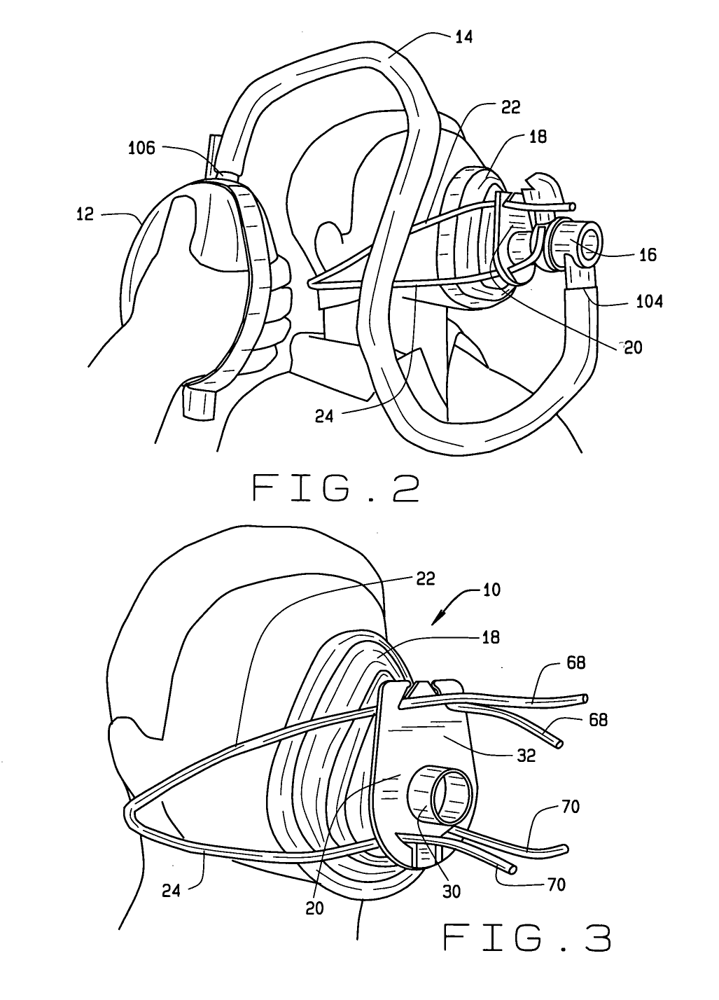

Referring to the drawings, an embodiment of a mask restraining system used with a bag mask resuscitator 9 is illustrated and generally indicated as 10 in FIGS. 2-8. In one embodiment, the bag mask resuscitator 9 may include a hollow, flexible tubing 14 of suitable length and flexibility having a proximal end 106 engaged to a resuscitation bag 12 and a distal end 104 attached to a valve assembly 16 which is rotatably coupled to a mask 18 adapted for placement on a patient's face. However, it is contemplated that the mask restraining system 10 may be used with a bag mask resuscitator 9 having the resuscitation bag 12 in direct engagement with the valve assembly 16 without the flexible tubing 14 being attached. As used herein the term “distal” means the end farthest away from the user, while “proximal” means the end closest to the user.

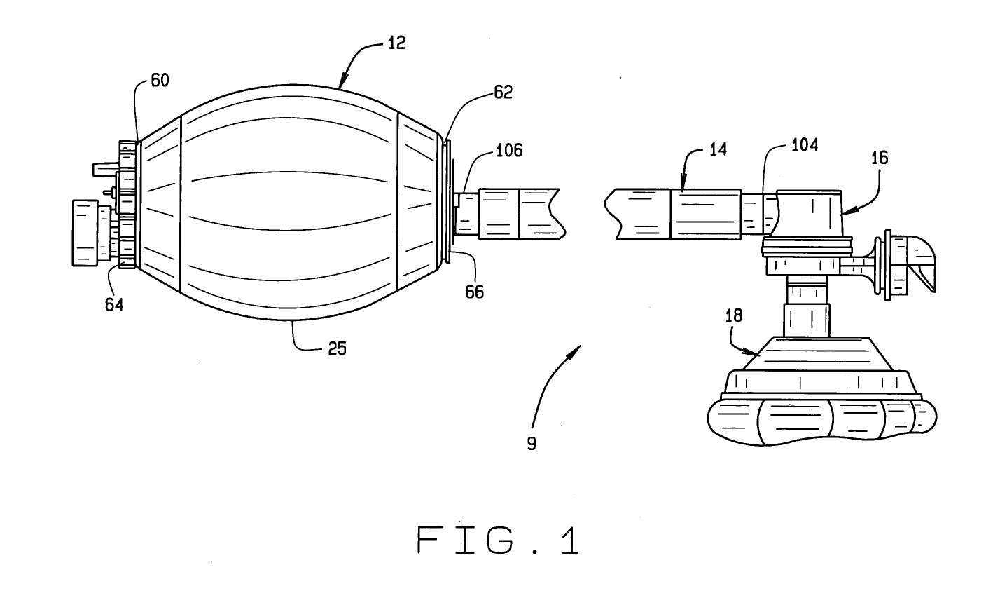

Referring to FIGS. 1 and 2, the resuscitation bag 12 may provide a means for forcing air into a patient's respiratory system through the flexible tubing 14 by repeated actuation of the resuscitation bag 12. The resuscitation bag 12 may include a flexible, hollow body 25 defining a chamber (not shown) that communicates with a proximal opening 60 and an opposing distal opening 62. The resuscitation bag 12 may further include an inlet manifold 64 and an outlet manifold 66 which are disposed along the proximal and distal openings 60, 62, respectively, for acting as connection sites as well as regulating fluid flow through the resuscitation bag 12. In addition, the resuscitation bag 12 may include a main valve flapper (not shown) engaged to a main valve retainer (not shown) that regulates the direction of fluid flow through the main valve retainer when the resuscitation bag 12 is actuated by the user.

As noted above, the flexible tubing 14 is connected between the valve assembly 16 and the resuscitation bag 12 and may be of sufficient length and flexibility that, in combination with the freely rotatable coupling of the valve assembly 16 with the mask 18, forces generated by actuation of the resuscitation bag 12 are isolated and are not applied to the mask 18. This is due to the fact that the flexible tubing 14 in combination with the rotational coupling of the valve assembly 16 prevent such forces from affecting the placement of the mask 18 on the patient's face.

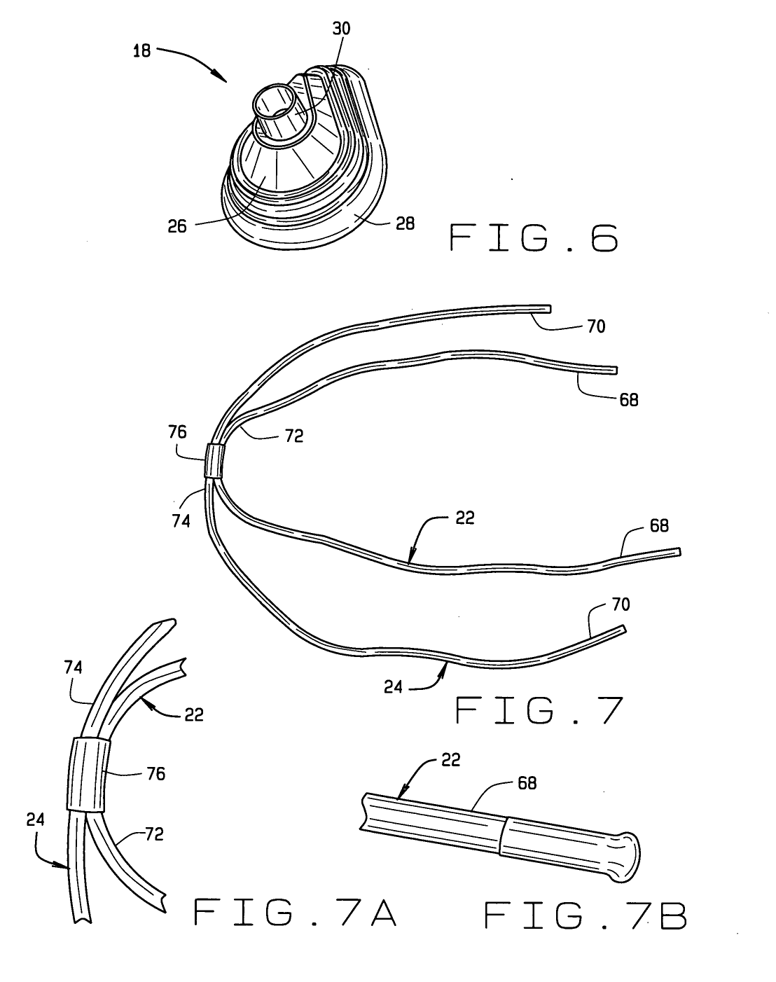

Referring to FIG. 6, mask 18 may include a mask portion 26 defining a port 30 adapted to engage the valve assembly 16 such that the valve assembly 16 freely rotates relative to port 30 of mask 18, thereby isolating any forces applied to the valve assembly 16 by flexible tubing 14. The mask 18 may further include a seal portion 28 along the periphery of mask portion 26 to provide a sufficiently air tight seal between the mask 18 and the patient's face.

A more detailed description of the operation and structure of the bag mask resuscitator 9 for use with the mask restraining system 10 is provided in U.S. Provisional Patent Application Ser. No. 60/656,176 filed on Feb. 25, 2005 and is herein incorporated by reference in its entirety.

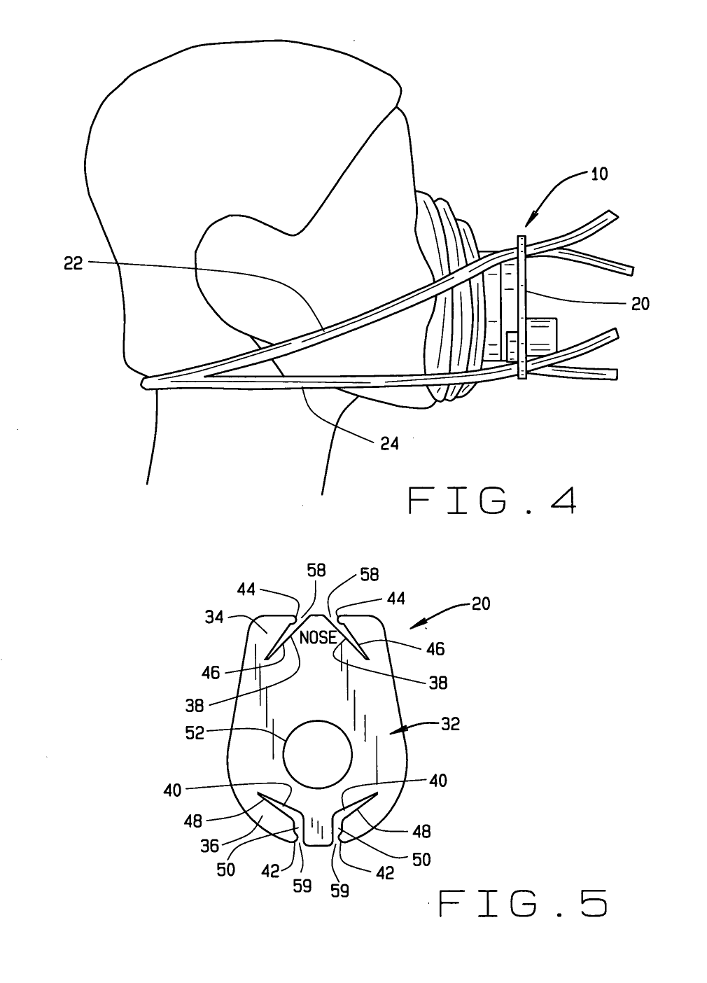

Referring back to FIGS. 2 and 4, the mask restraining system 10 may include a retainer 20 adapted to engage a first cord 22 and a second cord 24 as well as the mask 18 on the patient's face. As shown in FIG. 5, retainer 20 may include a retainer body 32 defining an upper portion 34 and a lower portion 36. The retainer body 32 may define an aperture 52 adapted to receive the port 30 of mask 18 therethrough when engaging retainer 20 to mask 30 as shall be discussed in greater detail below.

As further shown, the upper portion 34 of retainer body 32 defines a pair of upper slots 38 that may engage first cord 22, while lower portion 36 defines a pair of lower slots 40 that may engage second cord 24. However, it is contemplated that either pair of upper slots 38 or lower slots 40 may engage either the first cord 22 or second cord 24 which are identical in structure.

In addition, the pair of upper slots 38 may be defined such that each upper slot 38 has a single angled slot formed in diametric opposition to the other upper slot 38. The pair of upper slots 38 may each include a progressively narrowing pinch wedge 46 that communicate with a respective entrance 58 which defines a protrusion 44. Protrusion 44 is adapted to retain the first cord 22 within each upper slot 38 after the first cord 22 has been inserted through the respective entrance 58 by partially blocking entrance 58.

As shown, each lower slot 40 may be defined to have a double angled slot formed in diametric opposition to the other lower slot 40. In addition, each lower slot 40 may define an entry portion 50 that communicates with a progressively narrowing pinch wedge 48. Each entry portion 50 communicates with an entrance 59 that defines a protrusion 42. Similar to protrusion 44, protrusion 42 provides the same retaining function as protrusion 44 such that the second cord 24 does not become inadvertently disengaged from the lower slot 40 through entrance 59.

In one embodiment, retainer body 32 may have a slightly tapered shape wherein the upper portion 34 has a narrower width than the lower portion 34. However, it is contemplated that retainer body 32 may have other configurations, such as square, round, rectangular, reverse tapered and/or angular.

Referring to FIG. 7, first cord 22 may have the same material, structure and shape as second cord 24 and include opposite end portions 68 with a middle portion 72 defined between end portions 68. Similarly, second cord 24 may also include opposite end portions 70 with a middle portion 74 defined between portions 70. As shown in FIG. 7A, in one embodiment the middle portions 72, 74 of the first and second cords 22, 24 may be bound together by a shrink tube 76. During manufacture, the shrink tube 76 surrounds a portion of both middle portions 72, 74 and then a hot air process may be used to shrink the shrink tube 76 such that the middle portions 72, 74 are bound together.

In addition, the end portions 68, 70 may be dipped in a PVC plastisol protective covering as illustrated in FIG. 7B. In one embodiment, first and second cords 22, 24 may be made of a flexible and/or elastic material, such as materials made to manufacture a bungee cord, although other flexible and/or elastic materials are contemplated.

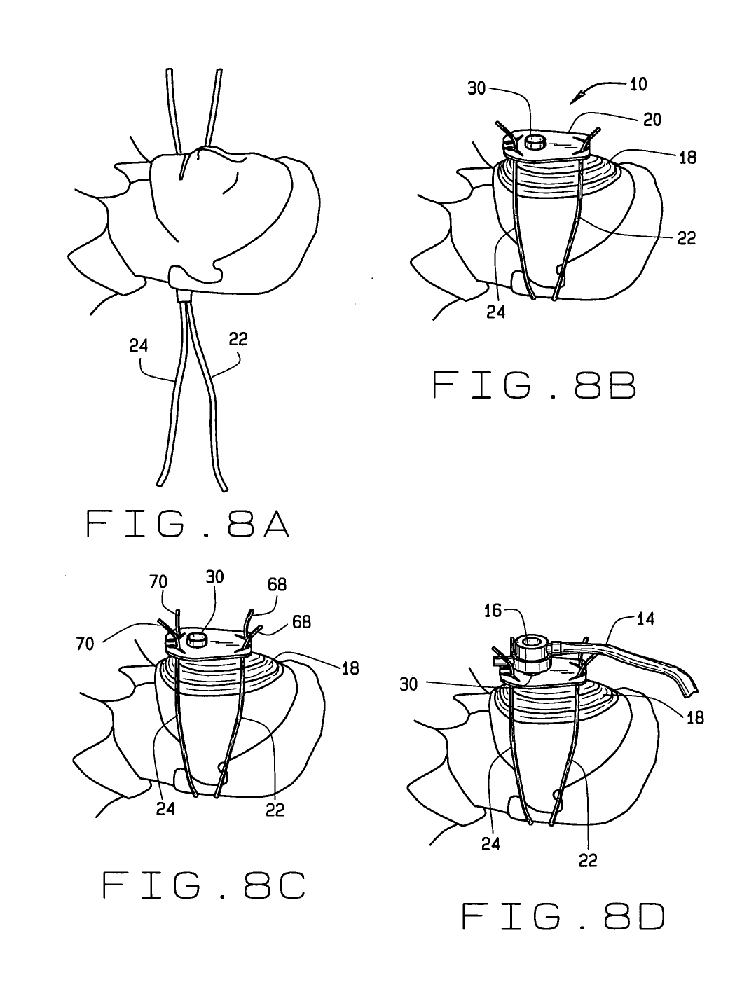

Referring to FIGS. 8A-8D, one embodiment of the method for utilizing the mask restraining system 10 is illustrated. As shown in FIG. 8A, the middle portions 72, 74 of first and second cords 22, 24 may be passed behind the head of a patient generally along the bridge of the neck such that the head of the patient does not need to be moved in order to pass the first and second cords 22, 24 around the neck. Once the first and second cords 22, 24 are so positioned, the mask 18 is engaged to the retainer 20 by inserting the port 30 of mask 18 through the aperture 52 of the retainer body 32 such that the mask 18 may then be placed on the face of the patient as illustrated in FIG. 8B.

After placement of the mask 18, the first cord 22 may be engaged to the retainer 20 by attaching the end portions 68 to respective upper slots 38, while then attaching the end portions 70 of second cord 24 to respective lower slots 40. It is contemplated that either the first cord 22 or second cord 24 may be attached first to the retainer 20. The mask 18 may then be adjusted and the first and second cords 22, 24 pulled upwardly relative to retainer body 32 in tugging action illustrated in FIG. 8C which tightens the mask 18 onto the patient's face. Once the first and second cords 22, 24 are tightened, the mask 18 is securely fitted on the patient's face as shown in FIG. 8D.

As noted above, the method for securely attaching the mask restraining system 10 requires the first cord 22 be engaged with the upper slots 38 such that the end portions 68 are inserted through each entrance 58 and engage the progressively narrowing pinch wedge 46 in a pinching action as the first cord 22 is pulled upwardly relative to the retainer body 32. The pinching action of pulling the end portions 68 of the first cord 22 upwardly through the progressively narrowing pinch wedge 46 generates a pinching action that engages each end portion 68 with each respective upper slot 38.

The second cord 24 may then engaged to the lower slots 40 such that the end portions 70 of second cord 24 are first inserted through each entrance 59 of lower slots 40 and into respective entry portions 50. In this engagement with each entry portion 50, the second cord 24 is not fully engaged and may be further tightened relative to the mask 18. This is accomplished as shown in FIG. 8C by engaging each end portions 70 with the respective progressively narrowing pinch wedge 48 by pulling upwardly relative to the retainer body 32 until a pinching action is generated that engages each end portion 70 with the respective lower slot 40. In this manner, the first cord 22 and second cord 24 tighten the mask 18 securely against the patient's face.

It should be understood from the foregoing that, while particular embodiments have been illustrated and described, various modifications can be made thereto without departing from the spirit and scope of the invention as will be apparent to those skilled in the art. Such changes and modifications are within the scope and teaching of this invention as defined in the claims appended hereto.

What is claimed:

1. A mask restraining system comprising:
a retainer, said retainer having a retainer body defining an aperture, said retainer body further defining an upper portion and a lower portion, said upper portion defining a pair of upper slots, while said lower portion defines a pair of lower slots; and
a first cord and a second cord each being adapted to engage said retainer body,
wherein said upper slots define a progressively narrowing pinch wedge adapted to engage either said first cord or said second cord in a pinching action and said lower slots define a progressively narrowing pinch wedge adapted to engage another of said first cord or said second cord in a pinching action.

2. The mask restraining system according to claim 1, wherein said upper slots define a pair of opposite single angled slots, each of said opposite single angled slots being in communication with an entrance.

3. The mask restraining system according to claim 2, wherein said lower slots define a pair of opposite double angled slots, each of said pair of opposing double angled slots being in communication with an entrance.

4. The mask restraining system according to claim 2, wherein each of said entrances define a protrusion.

5. The mask restraining system according to claim 3, wherein each of said entrances define a protrusion.

6. The mask restraining system according to claim 1, wherein said first cord and second cord are a bungee cord.

7. The mask restraining system according to claim 1, wherein said first cord and said second cord include opposite end portions wherein each of said opposite end portions is adapted to engage said upper slots and said lower slots in a manner that permits said middle portions of said first cord and said second cord to be tightened towards said retainer body.

8. The mask restraining system according to claim 2, wherein said opposite single angled slots define said progressively narrowing pinch wedge.

9. The mask restraining system according to claim 3, wherein said opposite double angle slots include an entry portion in communication with said progressively narrowing pinch wedge.

10. The mask restraining system according to claim 9, wherein said entry portion is at a different angle relative to said progressively narrowing wedge.

11. A resuscitator apparatus comprising:
a resuscitation bag in fluid flow communication with a mask seated on the face of a patient, wherein said mask defines a port, and
a mask restraining system adapted to engage said mask in a tightening action, said mask restraining system comprising a retainer, said retainer including a retainer body defining an aperture, said aperture being adapted to engage said port of said mask, said retainer body further defining an upper portion and a lower portion, said upper portion defining a pair of upper slots, while said lower portion defines a pair of lower slots, and a first cord and a second cord, wherein said upper slots are adapted to engage said first cord in a pinching action while said lower slots are adapted to engage said second cord in a pinching action such that a tightening action is applied to said mask by said retainer body.

12. The bag mask resuscitator according to claim 11, wherein first cord and said second cord define a middle portion adapted to engage the patient.

13. The mask restraining system according to claim 11, wherein said upper slots define a pair of opposite single angled slots, each of said opposite single angled slots being in communication with an entrance.

14. The mask restraining system according to claim 13, wherein said lower slots define a pair of opposite double angled slots, each of said pair of opposing double angled slots being in communication with an entrance.

15. The mask restraining system according to claim 13, wherein each of said entrances define a protrusion.

16. The mask restraining system according to claim 14, wherein each of said entrances define a protrusion.

17. The mask restraining system according to claim 11, wherein said first cord and second cord are a bungee cord.

18. The mask restraining system according to claim 11, wherein said first cord and a second cord include opposite end portions, wherein each of said opposite end portions is adapted to engage said upper slots and said lower slots in a manner that permits said middle portions of said first cord and said second cord to be tightened towards said retainer body.

19. The mask restraining system according to claim 13, wherein said opposite single angled slots define a progressively narrowing pinch wedge.

20. The mask restraining system according to claim 14, wherein said opposite double angle slots include an entry portion in communication with a progressively narrowing pinch wedge.

21. The mask restraining system according to claim 20, wherein said entry portion is at a different angle relative to said progressively narrowing wedge.

22. A method of using a mask restraining system comprising:
a) providing a mask restraining system comprising a retainer, said retainer comprising a retainer body defining an aperture, said retainer body further defining an upper portion and a lower portion, said upper portion defining a pair of upper slots, while said lower portion defines a pair of lower slots, said upper slots each defining a progressively narrowing pinch wedge and said lower slots each defining an entry portion in communication with a progressively narrowing pinch wedge, said upper slots adapted to engage a first cord having a middle portion defined between opposite end portions and said lower slots adapted to engage a second cord having a middle portion defined between opposite end portions;
b) engaging the port of said mask with the aperture of said retainer body;
c) passing the first cord and the second cord behind the neck of a patient;
d) engaging the end portions of said first cord to said upper slots and engaging the end portions of said second cord to said lower slots; and
e) pulling the end portions of said first cord and said second cord away from said retainer body such that said first cord and said second cord are engaged to said upper slots and said lower slots, respectively, in a pinching action.

23. The method of using a mask restraining system according to claim 22, wherein the step of positioning said first cord and said second cord behind the bridge of the neck of the patient includes positioning the middle portion of each of said first cord and said second cord behind the bridge of the neck of the patient.

24. A mask restraining system comprising:
a retainer, said retainer comprising a retainer body defining an aperture, said retainer body defining an upper portion and a lower portion, said upper portion defining a pair of upper slots, while said lower portion defines a pair of lower slots, each of said upper slots defining a progressively narrowing pinch wedge while each of said lower slots define an entry portion in communication with a progressively narrowing pinch wedge, said upper slots being adapted to frictionally engage a first cord and said lower slots being adapted to frictionally engage a second cord.

25. A mask restraining system comprising:
a retainer, said retainer comprising a retainer body defining an aperture, said retainer body further defining an upper portion and a lower portion, said upper portion defines a pair of upper slots, while said lower portion defines a pair of lower slots; and
a first cord and a second cord, said first cord and said second cord being adapted engage said upper slots and said lower slots, respectively, in a pinching action.
